(19) United States
(12) Patent Application Publication
(10) Pub. No.: US 2026/0210341 A1
(43) Pub. Date: Jul. 23, 2026

(54) HEATING FOR MEMORY METAL SHAPE-SETTING

(71) Applicant: Edwards Lifesciences Corporation, Irvine, CA (US)

(72) Inventors: Jonathan C. MacWilliams, Sandy, UT (US); Daniel Arthur Payne, Herriman, UT (US); Cameron Frank Sullivan, Saratoga Springs, UT (US)

(21) Appl. No.: 19/572,684

(22) Filed: Mar. 19, 2026

Related U.S. Application Data
(63) Continuation of application No. PCT/US2024/047431, filed on Sep. 19, 2024.
(60) Provisional application No. 63/584,312, filed on Sep. 21, 2023.

(51) Int. Cl. F03G 7/06; A61F 2/91; A61L 31/14; H05B 6/10; H05B 6/36

(52) U.S. Cl. CPC F03G 7/0614; A61F 2/91; A61L 31/14; H05B 6/105; H05B 6/36; A61F 2210/0038; A61L 2400/16

(57) ABSTRACT

A shape-setting device comprising an electrical conductor and a shaping form configured to have a target memory-metal device physically coupled thereto to hold at least a portion of the target memory-metal device in a shape conforming to at least a portion of the shaping form. A method of shape-setting a memory-metal device includes physically-coupling a memory-metal device to a shaped workpiece, injecting electrical current through a conductor associated with the shaped workpiece to heat the shaped workpiece, and conducting thermal energy from the shaped workpiece to the memory-metal device to shape-set at least a portion of the memory-metal device to a shape conforming to at least a portion of the shaped workpiece.

### RELATED APPLICATION(S)

This application is a continuation of PCT Patent Application No. PCT/US2024/047431 filed on Sep. 19, 2024 which claims priority to U.S. Provisional Patent Application Ser. No. 63/584,312, filed on Sep. 21, 2023 and entitled HEATING FOR MEMORY METAL SHAPE-SETTING, the complete disclosure of each of these applications being hereby incorporated by reference in its entirety.

### BACKGROUND

The present disclosure generally relates to the field of shape-memory devices and processes. In accordance with some applications, shape-memory devices, which can include medical implant devices (e.g., stent implant devices) can be shape-set to a desired shape by heating the device structure in accordance with an annealing/shape-setting process. Shape-setting heating can be implemented by immersing the target structure in a molten salt bath. Molten salt bath systems can be relatively expensive, massive, and complex systems.

### SUMMARY

Described herein are devices, methods, and systems relating to heating memory-metal devices to a shape-setting temperature using tools/devices configured to generate heat using electrical current in one or more conductors. Such conductor(s) may be associated with a shaping form/workpiece configured to shape the target memory-metal device while heating the device using the generated heat. Conductors used for heating in example shape-setting devices of the present disclosure can comprise inductive coils, wherein heat is generated in the shaping workpiece and/or target device using induced heat produced by injecting a current (e.g., alternating current) in the conductor to induce current in the workpiece or target device that produces resistive heating.

In some examples, heat is generated at least in part due to resistive heating effects of the conductor itself from the current passing therethrough. Conductors of shape-setting devices disclosed herein can be embedded within a volume of the associated shaping workpiece, or may be disposed within and/or without the workpiece form. In some examples, the conductor itself is shaped to provide the shaping form of the device/tool. Furthermore, examples of the present disclosure may have structural conductor and/or shaping workpiece features (e.g., variable thickness, spacing, material composition, etc.) that produce a variable heating profile over different areas of the device.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 shows a shape-memory implant device implanted in example anatomy in accordance with some examples.

FIG. 2 is a flow diagram illustrating a process for shape-setting a memory-metal device using immersion-bath heating in accordance with some examples.

FIG. 3A shows a memory-metal device disposed on a shaping mandrel in accordance with one or more examples.

FIG. 3B shows a memory-metal shape-setting immersion bath system in accordance with one or more examples.

FIG. 4 is a block diagram of an induction-coil heating system for memory-metal shape-setting in accordance with one or more examples.

FIG. 5A shows a side view of a shaping workpiece having an induction coil integrated therewith in accordance with one or more examples.

FIGS. 5B and 5C show side cross-sectional views of implementations of the shaping workpiece of FIG. 5A, which has an induction coil integrated therewith, in accordance with one or more examples.

FIG. 6A shows a side view of a shaping workpiece having an induction coil associated therewith in accordance with one or more examples.

FIGS. 6B and 6C show side cross-sectional views of implementations of the shaping workpiece of FIG. 6A, which has an induction coil associated therewith, in accordance with one or more examples.

FIGS. 7A and 7B show perspective and side views, respectively, of a multi-piece shaping workpiece in accordance with one or more examples.

FIG. 7C shows a side cross-sectional view of a multi-piece shaping workpiece having an induction coil integrated with one or more interior pieces thereof in accordance with one or more examples.

FIG. 7D shows a side cross-sectional view of a multi-piece shaping workpiece having an induction coil integrated with one or more exterior pieces thereof in accordance with one or more examples.

FIG. 8 shows a side cross-sectional view of a multi-piece shaping workpiece having an induction coil associated with an inner and/or outer diameter thereof in accordance with one or more examples.

FIG. 9 shows perspective and side views of a piece of a multi-piece shaping workpiece that has a dedicated/corresponding induction coil portion associated therewith in accordance with one or more examples.

FIG. 10 is a block diagram of a resistive heating system for memory-metal shape-setting in accordance with one or more examples.

FIGS. 11A and 11B show a resistive heating workpiece formed of one or more electrical conductors in accordance with one or more examples.

FIG. 12 is a flow diagram illustrating a process for heating a memory metal device using a heating tool in accordance with one or more examples.

FIG. 13 shows a heating workpiece configured to produce memory metal shape-setting variability in accordance with one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example. Furthermore, where a reference identifier is used that comprises a first numeric portion followed by a dash (e.g., ‘-’) and a second numeric portion (e.g., ‘10-2,’ where ‘10’ is the first numeric portion and ‘2’ is the second numeric portion), references in the written description to only the first numeric portion (e.g., ‘10’) may refer to any feature identified in the figures using such first numeric portion (e.g., ‘10-1,’ ‘10-2,’ ‘10-3,’ etc.), even where such features are identified with reference identifiers that concatenate the numeric portion thereof with a dash and a second numeric portion. That is, a reference in the present written description to a feature ‘10’ may be understood to refer to either an identified feature ‘10-1’ in a particular figure of the present disclosure or to an identifier ‘10’ or ‘10-2’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Shape-Memory Implants/Devices

Solutions of the present disclosure relate to shape-memory devices, and in particular to systems, devices, and methods for shape-setting shape-memory devices through heating. The term “shape-memory” is used herein according to its broad and ordinary meaning and can refer to devices, structure, or the like, having the ability to return to a predefined shape when subjected to certain stimulus, such as heat, electricity, light, and/or magnetic fields. Shape memory devices disclosed herein can include shape-memory alloys, shape-memory polymers, shape-memory ceramics/composites, and/or devices comprising any other type of shape-memory material. Shape-memory devices disclosed herein can comprise memory-metal devices, which generally include shape-memory devices made from shape-memory alloys, such as nitinol, a nickel-titanium alloy. Nitinol can be considered well-suited for use in medical devices, such as stent implants, frames, and/or other implant devices disclosed in detail herein, due to its biocompatibility characteristics. Other memory metals that can be used in connection with examples of the present disclosure include copper-aluminum-nickel (CuAlNi) alloy, copper-zinc-aluminum (CuZnAl) alloy, iron-manganese-silicon (FeMnSi) alloy, nickel-aluminum (NiAl) alloy, platinum-cadmium (PtCd) alloy, gold-cadmium (AuCd) alloy, and others. The terms “shape-memory” and “memory-metal” are used interchangeably herein in some contexts.

As referenced above, shape-memory implants (e.g., stents, docking frames, prosthetic valve frames, etc.) relevant to solutions presented herein can advantageously be configured to return to an original shape-set shape/form upon heating, or in response to another stimulus, after being deformed. Shape-memory materials can provide certain functionality/feature(s) that make the use of such materials attractive for implant devices/structures and other applications. For example, shape-memory devices generally provide a shape-memory effect, wherein when the material/metal is deformed at a low temperature, the material/metal has a tendency to return to its original, shape-set shape when heated or otherwise stimulated. Therefore, shape-memory implants/devices can advantageously change shape in response to changes in temperature.

In addition to shape-memory effect, some shape-memory devices can provide superelasticity or pseudoelasticity. For example, for some shape-memory materials/metals, when deformed at a certain temperatures, the device/structure can revert back to its original shape without the need for heating. Such functionality can be useful in applications that require a large amount of elastic deformation. Certain shape-memory materials/metals, such as nitinol, further provide biocompatibility characteristics, making them particularly suitable for use in medical devices, such as stents. Shape-memory devices associated with examples of the present disclosure can be used in any suitable or desirable application. For example, shape-memory devices of the present disclosure can be used for medical devices, as described in detail below, as well as devices used for aerospace, automotive, and other industries.

As referenced above, shape-memory devices shape-set in connection with various examples herein can be any type of implant, or non-implant, devices. Example implant devices that may be shape-set according to aspects of the present disclosure can include shape-memory/metal heart valve frames, heart valves, blood vessel (e.g., aortic, coronary artery) stents, neurological/brain stents, cardiac valve (e.g., mitral) repair devices, cardiac valve (e.g., pulmonic/mitral) docking stations, suture clips, blood vessel (e.g., inferior vena cava) filters, ventricle remodeling devices (e.g., chainmail pouches, tethers), guidewires (e.g., shape-memory pigtail features), and/or any other shape-memory implant devices/structures.

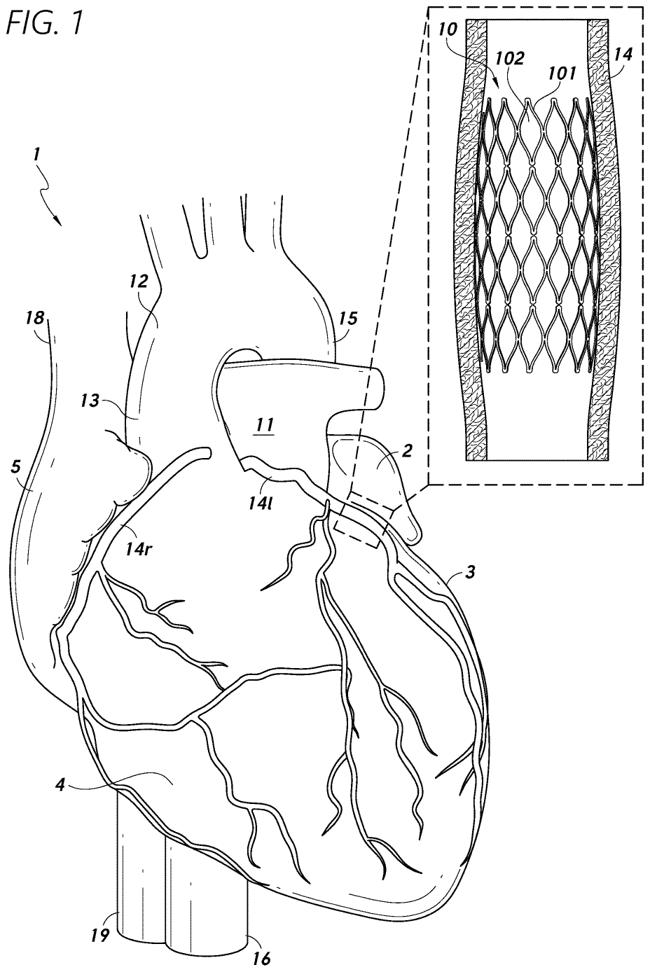

Certain examples are disclosed herein in the context of memory-metal blood vessel stents, such as stents implantable in a coronary artery or other arterial or venous blood vessel. FIG. 1 shows an example shape-memory implant device 10 implanted in example cardiac anatomy. The implant device 10 is an example device that may be shape-set in accordance with heating solutions disclosed herein.

FIG. 1 shows an example mammal heart 1. The blood circulation facilitated by the heart 1 generally includes coronary circulation, pulmonary circulation, and the larger systemic circulatory system, each including certain venous and arterial blood vessels. The systemic circulatory system is supplied by oxygenated blood pumped from the heart into certain arteries, the largest of which is the aorta 16, which originates from the left ventricle 3 of the heart 1 and serves to carry and distribute oxygen-rich blood to all parts of the body. The aorta 16 is divided into the ascending aorta 13, the aortic arch 12, and the descending aorta 15, which further splits into the thoracic aorta (in the chest) and the abdominal aorta.

Venous blood returns to the heart primarily via the superior 18 and inferior 19 vena cavae, which are the largest veins in the body. The superior vena cava 18 returns deoxygenated blood from the upper half of the body to the right atrium 5 of the heart 1, whereas the inferior vena cava 19 carries deoxygenated blood from the lower half of the body back to the heart 1, also emptying into the right atrium 5.

The pulmonary circulation provides the pathway of blood between the heart 1 and the lungs. Deoxygenated blood returning to the heart 1 via the superior 18 and inferior 19 vena cavae enters the right atrium 5 and moves into the right ventricle 4, which pumps the blood to the lungs via the pulmonary arteries 11. In the lungs, the blood becomes oxygenated and returns to the heart 1 via the pulmonary veins, entering the left atrium 2 and then the left ventricle 3.

The heart 1 includes a system of blood vessels forming the coronary circulation, which supplies the heart muscle 1 itself with oxygen and nutrients. The coronary circulation includes various coronary arteries 14, which supply the heart muscle, or myocardium, with oxygenated blood. The two main coronary arteries include the left 14l and right 14r coronary arteries. The left coronary artery 14l divides into the left anterior descending artery and the circumflex artery. The right coronary artery 14r typically supplies the right side of the heart and often the lower portion of both ventricles and the posterior heart structures. When the left ventricle 3 contracts, oxygen-rich blood is pushed into the aorta 16, which distributes the blood throughout the body via the systemic circulation. At the same time, some of the oxygenated blood from the aorta 16 is directed to the heart muscle itself via the coronary arteries 14. After delivering oxygen to the heart muscle, this blood drains into the coronary sinus, which empties into the right atrium, joining the deoxygenated blood from the rest of the body.

Shape-memory stent devices, such as the stent 10, can comprise a flexible tubular structure made of memory metal or other shape-memory material that is configured to be inserted into a target blood vessel or other bodily canal, wherein the stent 10, once deployed, can serve to hold the target blood vessel segment in an open state to allow blood flow therethrough. Shape-memory stent devices can be implanted for the treatment of certain cardiovascular conditions. For example, coronary artery stents, such as the stent 10, can be used in an angioplasty procedure to treat coronary artery disease, which is the narrowing or blockage of the coronary arteries due to a build-up of plaque. When the coronary arteries 14 are narrowed or blocked, symptoms including chest pain (angina), or a heart attack can result.

The stent implant 10 can be advanced to the target blood vessel segment in a delivery system/catheter, which may access a downstream target blood vessel segment via percutaneous arterial (and/or venous) entry (e.g., in the groin or wrist). The delivery system may hold the stent 10 in a radially-compressed delivery configuration while the delivery system is guided towards the target blood vessel segment (e.g., narrowed or blocked coronary artery). The delivery system/sheath may have a small balloon at its tip, which can be inflated once the delivery system is in place to expand the stent 10 to compress the plaque against the artery wall and restore/improve blood flow. The shape memory of the stent 10, which may be set to the expanded configuration of the stent, can additionally or alternatively produce the desired stent expansion in the blood vessel 14. When expanded from the compressed delivery configuration, the struts 101 of the stent 10 may move apart, increasing the size of the cells 102 and causing the stent 10 to conform to the size and shape of the blood vessel 14. Coronary artery stents can provide a less invasive alternative to coronary artery bypass grafting.

In some implementations, the stent 10 may be coated with medication/substance that is slowly released from the stent and helps to prevent the target blood vessel from becoming blocked again. Such medication may impede the formation of scar tissue that may otherwise form in the stent and cause the blood vessel to narrow again.

As shown and referenced above, shape-memory stents, such as the stent 10 of FIG. 1, can have a metal frame of struts 101 forming open cells 102. Such designs can provide desirable flexibility, conformability, and scaffolding support to keep the target blood vessel open. Generally, the struts 101 provide the individual forms that make up the structure of the stent. The struts 101 can be formed of thin, elongated pieces of metal that connect together to form the overall structure of the stent frame. The stent frame may have a generally tubular shape to conform to the cylindrical shape of the target blood vessel. The stent frame can advantageously be designed to be flexible so it can navigate the often tortuous paths of the blood vessels in a radially-compressed delivery configuration, but also rigid enough to provide the necessary support to keep the blood vessel open. The open cells 102 of the stent 10 can be geometric in shape (for example, they may be diamond-shaped or circular) and evenly distributed across the stent structure. The open-cell design allows for flexibility while providing even support to the blood vessel wall, and can also enable the passage of blood into side branches.

The stent frame 10 can be formed by cutting the stent pattern into a shape-memory (e.g., nitinol) tube or wire, such as by a laser cutting process. Once cut, the stent 10 may be subjected to a series of finishing processes, such as electropolishing and/or cleaning processes, which may be implemented to remove any residues or contaminants.

Stents made from shape-memory materials, such as the stent 10, can provide certain advantages compared to traditional, non-shape-memory stents. The use of shape-memory material/metal can allow for the stent 10 to undergo deformation to compress the stent for delivery, wherein the pre-set expanded shape/configuration of the stent 10 is restored naturally upon heating of the stent 10 above its transformation temperature. The use of shape-memory material can also provide superelasticity or pseudoelasticity for the stent 10, which may allow the stent 10 to undergo significant deformation and immediately return to its original shape with or without an increase in temperature. In some examples, the superelastic properties of the stent 10 allow the stent 10 to flex and conform to the natural movement of the artery without causing damage, which can be particularly beneficial in locations where the blood vessel bends or flexes. Furthermore, the shape-memory characteristics of the stent 10 can provide self-expansion functionality for the stent 10, such that the stent 10 can be delivered to the target site in the compressed configuration (e.g., at a relatively cooler temperature) and then expand to its pre-set shape once the deployed and at body temperature. Self-expansion can allow for less traumatic and more precise placement of the stent 10. In addition, shape-memory material/metal, such as nitinol, is generally well-tolerated by the body, reducing the risk of inflammation or other adverse reactions. Once expanded, shape-memory (e.g., nitinol) stents can resist external compression better than some other types of stents, which can help maintain the vessel in a patent/open condition over time.

### Shape-Setting of Memory-Metal Devices

The present disclosure relates to processes for manufacturing shape-memory implant components, and specifically to particular processes for shape-setting/annealing nitinol stents or other implant components through heat treatment. Generally, devices comprising nitinol memory-metal nickel-titanium alloy, and other shape-memory alloys, can be heat-treated to cause the target component/device to ‘remember’ a particular shape that the metal has been set to during a shape-setting/annealing process. For example, a shape-set nitinol or other memory-metal form, when exposed to temperatures above an ‘activation temperature’ of the material, is prone to return/transition to its ‘remembered’ form/shape.

Shape-setting of memory-metal devices/materials in accordance with examples of the present disclosure can be considered and/or referred to as “annealing” in some contexts, wherein such term is used herein according to its broad and ordinary meaning. The terms “shape-setting” and “annealing,” as used herein, can be understood to refer to any heating process that alters the physical and/or chemical properties of a shape-memory material to set the shape memory behavior and/or transformation/activation temperatures of the shape-memory material (e.g., alloy), and/or to increase the material's ductility, reduce its hardness, or the like, to make the material more workable.

The shape-setting/annealing process for setting the shape of a memory-metal device generally involves heating the metal/alloy material to a relatively high temperature (e.g., around 500° C. (932° F.)) sufficient to cause the dynamic crystalline structure of the material to reorient to a more cubic crystalline structure conforming to the present shape of the metal when heated. During shape-setting/annealing, the memory-metal material can be held in the desired shape using a mandrel or similar shaping workpiece/tool in order to cause the device to remember the desired shape after shape-setting/annealing. After heating, when the device is cooled to a lower-temperature state (i.e., ‘martensite’ state), the device can be deformable from the cubic crystalline structure. Subsequent reheating above the activation temperature (which is lower than the annealing temperature, typically between 30° C. (86° F.) and 130° C. (266° F.)), wherein the specific activation temperature may be produced/set by the heating parameter(s), causes the memory metal to transition to its ‘austenite’ state, in which the crystalline structure of the metal becomes more cubic, causing reshaping to the previously-set cubic-structure shape thereof.

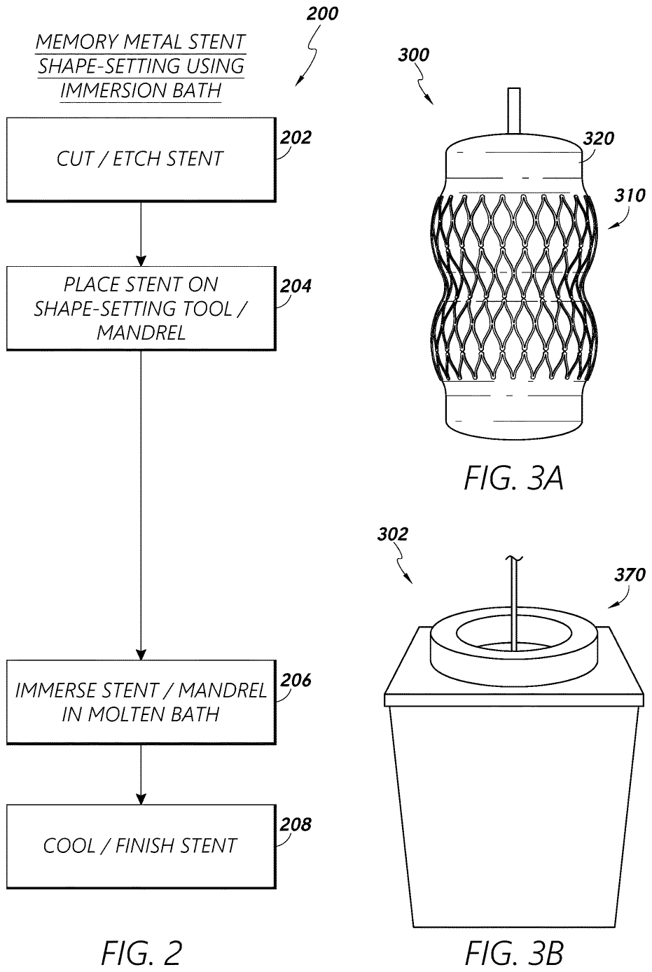

In some implementations, memory-metal shape-setting/annealing utilizes conduction heating through exposure/immersion in a superheated medium, such as a molten salt bath, to heat the material (e.g., nitinol), together with the shaping tool/mandrel on which the target device is held/disposed. FIG. 2 is a flow diagram illustrating a process 200 for shape-setting a memory-metal device using immersion-bath heating in accordance with some examples.

At block 202, the process 200 involves cutting/etching a stent frame, such as from a memory-metal tube or sheet, as described above, or other target memory-metal device/structure. The operation(s) associated with block 202 produce the pre-shape-set version of the stent/device. Prior to shape-setting/annealing, the stent may be relatively malleable, which condition may be suitable for stent frame cutting and/or forming.

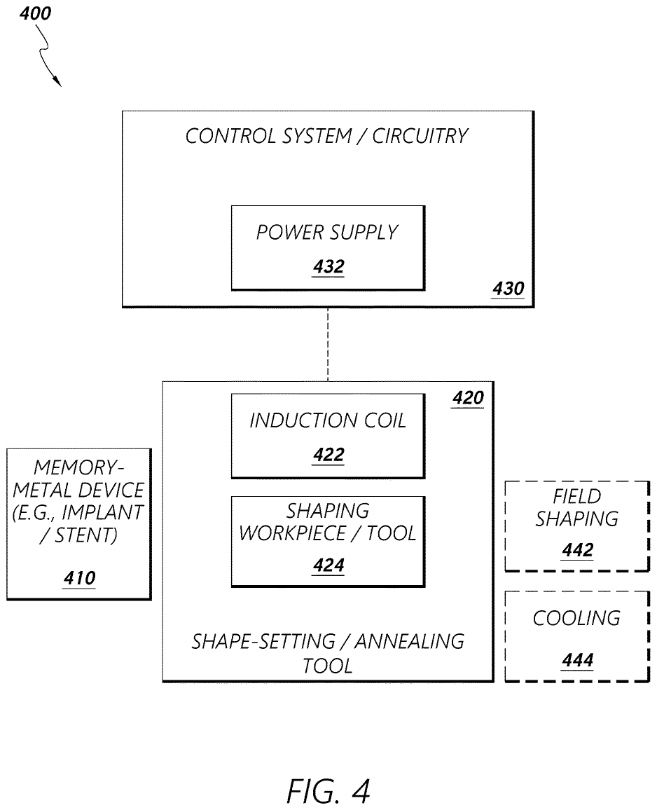

FIG. 3A shows a memory-metal device (e.g., stent) 310 disposed on a shaping mandrel/workpiece 320 in accordance with one or more examples. With further reference to FIGS. 2 and 3A, at block 204, the process 200 involves placing/disposing the stent 310 on the shape-setting tool/workpiece 320, which may be considered a mandrel in some contexts. For example, the shape-setting tool 320 may be shaped such that, when disposed on the tool 320, the stent 310 conforms to a shape thereof, wherein the shape of the mandrel 320 corresponds to a desired shape-setting configuration of the stent 310.

The shape-setting tool/workpiece 320 can be made of a heat-resistant material and designed to hold the stent 310 in the desired shape during the annealing process. In some examples, the tool/workpiece 320 comprises stainless steel, which may provide a suitably-high melting point for the heating process. The tool 320 can be designed to match the specific desired shape and dimensions of the stent 310 being produced. The tool/workpiece 320 may comprise a mandrel/mold form, which is used to form the stent 310 into the desired shape. For example, the tool 320 may be used to form the stent 310 into a specific diameter and/or to create certain complex shapes including variations in diameter over the length of the stent 10, as shown. The particular shape of the tool 320 and stent 310 in FIG. 3A is shows as a non-limiting example, and it should be understood that stents and other devices disclosed herein may have any suitable or desirable shape-set shape, including a straight cylinder, for example. The tool/workpiece 320 may further comprise feature(s) to help hold the stent 310 in place during the annealing process, such as clamps, pins, or other securing means. Such feature(s) can help ensure that the stent maintains its desired shape as it is heated and cooled.

The tool/workpiece 320 may consist of a single, unitary shaping form, or may comprise multiple pieces configured to combine to form the illustrated/described shaping form. Examples comprising a multipiece structure can facilitate molding the stent 310 to more complex geometries and/or allow for encapsulation of at least portion(s) of the stent 310.

At block 206, the process 200 involves immersing the stent 310 and mandrel/tool 320 together in a molten bath to heat the stent 310 for shape-setting/annealing. FIG. 3B shows a memory-metal-shape-setting immersion bath system 370 in accordance with one or more examples. Immersion of the stent 310 in the bath container of the system 370 can serve to heat the stent 310 to a specific temperature to induce a phase change, such that subsequent cooling ‘locks-in’ the desired shape of the stent 310. The molten salt bath system 370 can provide a controlled environment for the shape-setting/annealing process to take place, wherein the shape-setting tool 320 is used to create the desired shape of the stent 310 as the stent is heated.

The molten salt bath system 370 can comprise a container or tank made of a heat-resistant material, such as stainless steel, which is filled with a molten salt solution. The system can be configured to heat the molten salt solution to a specific temperature and hold at that temperature for a set amount of time to anneal the stent 310 to produce the desired properties thereof with respect to rigidity, activation temperature, etc. The system 370 may have any suitable or desirable dimensions. Furthermore, the heating medium may be any suitable heating medium. For example, in some implementations, the heating medium comprises a eutectic mixture of different salts, such as sodium chloride, potassium chloride, and/or lithium chloride. Such a mixture can advantageously provide a lower melting point than any of the individual salts. The particular composition of the molten salt can depend on the specific annealing requirements/properties of the stent 310. The molten salt bath can be heated using a heating element, such as a furnace or an electric heater. The temperature of the bath may be closely monitored and controlled to ensure that it remains at the desired temperature throughout the shape-setting/annealing process. The stent 310 is held in the bath for a set amount of time to allow for the formation of the desired crystalline structure, and then removed and allowed to cool.

The heat and time of the shape-setting/annealing process can affect various characteristics of the memory-metal stent 310, including its mechanical properties and/or shape memory behavior. For example, the particular heating temperature and heating time determine the tensile strength, ductility, and/or fatigue life of the stent 310. Generally, higher annealing temperatures and longer annealing times can increase the material's strength, while reducing its ductility. Furthermore, the heating temperature and/or time annealing process can affect the proportion of martensite and austenite crystal structures; higher annealing temperatures and longer annealing times can increase the amount of austenite crystal structure and decrease the amount of martensite crystal structure, which can affect the stent's shape-memory behavior.

Shape-setting/annealing temperature and/or time can further determine corrosion resistance characteristics of the stent 310. For example, the heating of the stent 310 during shape-setting/annealing can affect its surface properties and therefore its resistance to corrosion. Higher annealing temperatures and longer annealing times can lead to the formation of an oxide layer on the surface of the stent, which can improve its corrosion resistance. While described as ‘annealing’ in some contexts, it should be understood that shape-setting heating processes in accordance with the present disclosure may not involve heating a device to temperatures sufficient to relieve all stresses and/or redissolve any precipitates that may have formed during the manufacturing process, and therefore may not be considered a full metallurgical anneal according to some characterizations. The shape-setting heating process may be sufficient to achieve reorientation of the lattice of the atomic structure of the memory metal, but heating temperatures may or may not rise high enough to achieve metallurgical treatment beyond the structural reorienting of the lattice, such as grain growth, etc. Heating time in the immersion bath may be between 3-30 minutes, such as between 5-20 minutes, or other desirable period.

At block 208, the process 200 involves cooling and/or finishing the stent 310 to produce the final shape-memory product. After the annealing process is complete, the stent 310 may be removed from the bath and allowed to cool. In some implementations, the stent 310 may be cooled slowly in the molten salt bath (e.g., to room temperature) to provide control over the cooling process to prevent the formation of defects and to ensure that the annealing process is effective.

The stent 310 may be removed from the shape-setting tool/workpiece 320, and any excess material may be trimmed/polished off. For example, chemical polishing may be implemented to remove impurities, defects, residual stress, and/or oxide films, as desired. In some implementations, electropolishing may be performed according to a desirable voltage, current density, electrode distance, and time.

Compared to non-immersion heating solutions disclosed herein, the molten-bath heating process 200 can present undesirable complexity, cumbersomeness, and/or cost in some situations. For example, a molten-bath system can be relatively large, and heating using such systems can require several minutes to complete. Furthermore, the molten salt used for annealing memory-metal devices/stents can have a melting point of greater than 200° C.

### Induction and Resistive Heating of Shape-Memory Devices

As described in detail above, molten-salt-bath annealing can represent an undesirably expensive and slow process step for memory-metal device manufacturing, and can require significant space and energy. Furthermore, immersion shape-setting/annealing generally produces uniform metallurgic properties throughout the entire target device, rather than allowing for material variability to accommodate device needs/function. Disclosed herein are solutions for heating shape-memory devices using certain induction- and resistive-heating-based devices and systems, which can advantageously provide decreased size, time, and complexity solutions for shape-memory heating/setting, as well as improving safety and cleanliness conditions, compared to immersion-bath heating.

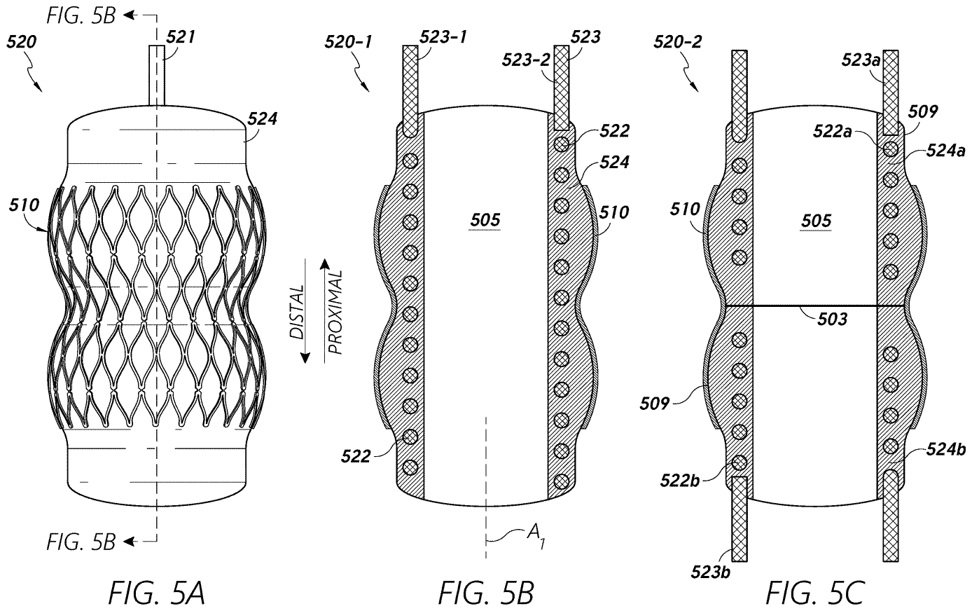

FIG. 4 is a block diagram of an induction-coil heating system 400 for memory-metal shape-setting in accordance with one or more examples. The system 400 is designed to implement induction heating using a coil 422 through which electrical current can be passed, such as high-frequency (e.g., radio-frequency (RF)) current, to induce heating in a workpiece 424. The system 400 can be configured to implement RF induction, magnetic induction, or other heating mechanism.

As an alternative to conduction-based, salt bath immersion heating for memory metal annealing, the system 400 is designed to use a shaping tool/workpiece 424 having an integrated/associated induction coil 422 used to induce current and heat for annealing/shape-setting a target memory metal structure/device 410 (e.g., stent) relatively quickly, safely, cleanly, and with less space requirement and equipment complexity compared to salt bath annealing. The induction coil 422 may be associated with the shaping tool/workpiece in any suitable or desirable manner. The terms “associated” and “associated with” are used herein according to their broad and ordinary meanings. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

Generally, the process of induction heating requires a coil that is used to induce electrical current in a metallic/conductive form, or ‘workpiece,’ to generate heat. Certain examples of the present disclosure advantageously combine the shaping tool/mandrel 424, which serves as an induction workpiece, and induction coil 422 into a single unit/device, thereby providing heating for shape-memory shape-setting/annealing and shaping in a single device/structure 420. The device 420 is designed to use magnetic, RF, or other induction modality to heat the shaping workpiece 424, or alternatively to induce current in the implant/stent 410 itself to cause the implant 410 to heat through direct resistive heating. The induction coil 422 is advantageously configured to generate sufficient heat in the workpiece 424 (or directly in the implant 410) to metallurgically transform the memory-metal part 410 to induce a new shape. The implant 410 may be any type of shape-memory implant, such as a component of a prosthetic valve (e.g., mitral), blood vessel or heart valve docking device (e.g., pulmonary valve dock), valve repair device/clip, delivery system, or any other device.

Disclosed induction-heating solutions combine the shape/function of a shape-setting tool/mandrel with an induction coil to produce a construction that promotes relatively rapid processing of memory metal shape-set components. The shape-setting tool 424 can have an at least partially cylindrical shape, for example. The induction coil tool 420 serves a dual purpose, to both heat and shape the memory-metal part 410 simultaneously.

In some implementations, induction heating involves using electromagnetic induction to heat electrically conductive materials. Such induced heating is generated by eddy currents, which are induced in the target material/workpiece by a rapidly changing magnetic field. Using induction heating as in the system 400 provides an alternative to the purely-conductive heating of salt bath systems, where heat transfer occurs when heat energy is transferred from the heated medium (i.e., salt bath) to the target through direct contact without inductive heating.

The heating system 400 includes the induction coil 422, which is powered by a power supply 432, which may comprise an electronic oscillator or other current source. In some implementations, the power supply 432 may generates a high-frequency alternating current that is passed through the induction coil 422, thereby producing a rapidly changing magnetic field that induces eddy currents in the workpiece 424, which may have the coil 422 at least partially embedded therein or otherwise disposed in physical proximity to the coil 422. The eddy currents may advantageously flow through volume of the workpiece 424, wherein the resistance of the workpiece material produces Joule/Ohmic/resistive heating. Generally, the amount of heat generated is proportional to the square of the current flowing through the material of the workpiece 424 and the resistance of the material.

The induction coil 422, which may be referred to as a ‘work coil’ or ‘heating coil,’ is a conductive coil comprising copper or other suitable conductor. The coil 422 can advantageously be designed and shaped to generate a high-frequency magnetic field. Furthermore, the coil may be shaped and positioned to direct the generated magnetic field toward the workpiece 424 to effectively induce heating in the workpiece. The shape and/or position of the induction coil 422 can at least partially determine the distribution of the magnetic field and influence the heating pattern caused in the workpiece 424 thereby. For example, the number of turns and geometry of the coil 422 can be designed to produce the desired strength and distribution of the magnetic field.

The system 400 can be configured to induce heating in the workpiece 424 using either magnetic or radio-frequency (RF) induction using the induction coil 422. For example, for magnetic induction, the frequency of the magnetic field generated by the coil 422 may be in the range of 10 kHz to 1 MHz. For RF induction, the magnetic field generated may be in the range of 1 MHz to 1 GHz. RF induction may be preferable in some solutions, as the higher-frequency field can provide a relatively more efficient heating process. Furthermore, while higher-frequency RF fields may not provide the depth of penetration of magnetic induction, depending on the configuration and position of the induction coil 422 relative to the workpiece 424, substantial depth of penetration (e.g., in the range of several centimeters) may not be necessary, and so RF field penetration may be suitable for shape-setting applications of the present disclosure in some implementations. Alternatively, where the workpiece is relatively thick, magnetic induction may be preferrable for induction-heating-based shape-setting as described herein. As RF induction heating can be more expensive than magnetic induction heating due to the complexity and cost of manufacture of the RF generator, magnetic induction may be preferrable despite potential inefficiencies.

The power supply 432 may be associated with a control system/circuitry 430 configured to regulate and/or monitor the heating process and/or perform certain temperature control and safety features. The power supply 432 can be configured to convert input power into the desired frequency and power level suitable for inductive heating of the workpiece 424. The power supply 432 can be, for example, solid-state or vacuum-tube-based, depending on the power requirements. In some implementations, the control system/circuitry 430 includes matching circuitry that matches the impedance of the power supply 432 to the induction coil 422 for efficient power transfer. In some implementations, the control system/circuitry 432 includes a capacitor bank configured to store electrical energy and provide relatively high-power bursts, as needed. Use of a capacitor bank can help enhance the efficiency of energy transfer to the workpiece 424 during the heating process, and can compensate for fluctuations in the power supply and improve the power factor. The control system/circuitry 430 can further include one or more temperature sensors, power controllers, feedback loops, user interfaces, and the like.

The terms “control circuitry” and “circuitry” are used herein according to their broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The workpiece form 424 comprises an electrically conductive material, which can include ferromagnetic (e.g., iron, steel) and/or non-ferromagnetic (e.g., aluminum, copper) material(s), at least in part. In examples comprising ferromagnetic workpiece components, heating of the workpiece 424 can be primarily caused by hysteresis losses resulting from alignment/realignment of magnetic domains within the material of the workpiece. With respect to non-ferromagnetic workpiece components, heating can be primarily through resistive losses induced by eddy currents within the material, as explained above. In some implementations, the workpiece 424 comprises at least one of steel, aluminum, copper, brass, or other conductive metal/alloy.

The system 400 may further include a cooling component/system 444, which may be configured to promote/maintain desirable temperatures of one or more of the system components. The cooling system 444 may be configured to employ, for example, water- or air-cooling methods to dissipate excess heat and prevent overheating.

The system 400 may further include certain magnetic/electric field shaping component(s) 442, which may be incorporated to shape and focus the magnetic/electric field produced by the induction coil 422. Such features may include magnetic concentrators, flux concentrators, magnetic shunts, and/or other features configured to improve/optimize the magnetic/electric field distribution and/or improve heating efficiency.

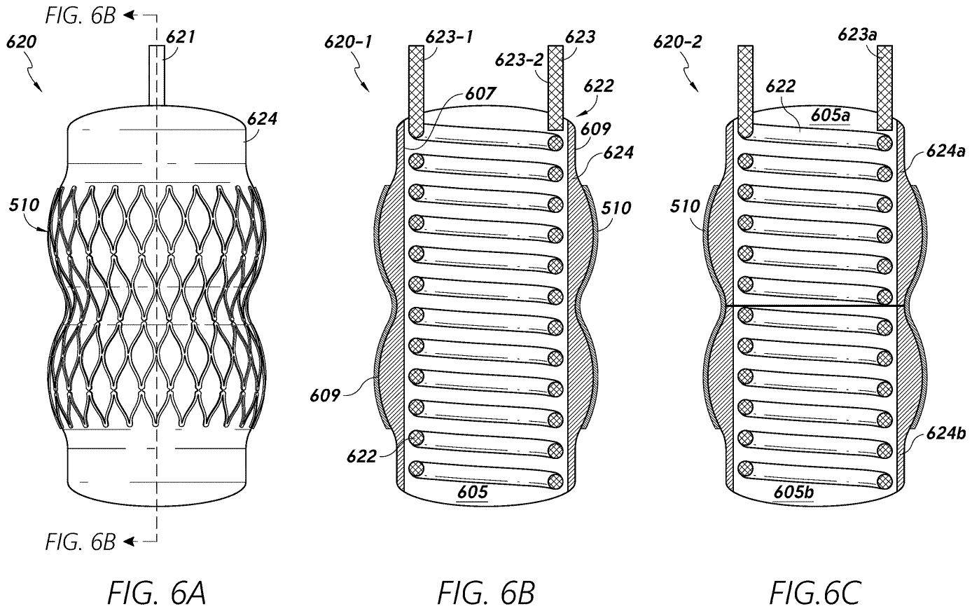

As referenced above, examples of the present disclosure can comprise shape-setting tools that include induction coil features integrated/built-into a shaping workpiece itself, which can provide a relatively simple, efficient tool for heating a memory-metal device (e.g., stent or other implant component). Such integrated coil/workpiece devices can have any suitable or desirable shape and/or configuration. FIG. 5A shows a side view of an integrated shape-setting tool 520 comprising a shaping workpiece 524 having an induction coil integrated therewith in accordance with one or more examples. FIGS. 5B and 5C provide side cross-sectional views of implementations of the integrated shape-setting tool/workpiece 520 of FIG. 5A, showing the integrated induction coil 522, in accordance with one or more examples. As with certain other examples disclosed herein, the conductor/coil 522 is disposed within an outer diameter 509 of the shaping form(s) 524.

FIG. 5A shows the shape-setting tool 520 with a memory-metal implant 510, such as a stent/frame, or similar, disposed about a shaping form 524 of the tool 520, such that the implant 510 generally conforms to the shape of the form 524 on one or more sides/areas thereof to produce a desired shape in the implant 510. Although certain examples are disclosed herein in the context of shaping forms for shape-setting tools that produce an hourglass-shaped stent or frame, it should be understood that such shape is shown for example illustration purposes only, and any example disclosed herein may be implemented with a shaping form having any suitable or desirable shape or configuration. Furthermore, shaping forms disclosed herein may have any number of pieces/segments, which may fit and/or work together to produce a desired form in the target implant.

In some implementations, the shape-setting tool/workpiece 520 includes certain structural supporting and/or handling features, such as one or more extensions 521, which may be separate from and/or integrated with certain electrical conductor components utilized for supplying/passing current through the induction coil 522 integrated and/or associated with the tool 520.

FIG. 5B shows a first example implementation 520-1 of the shape-setting tool/workpiece 520 shown in FIG. 5A. In the example of FIG. 5B, the induction coil 522 is integrated with a single-piece implementation of the shaping form 524. In some implementations utilizing single-piece shaping forms, such shapes may be straight cylinders or other shapes that are conducive to sliding a target implant/device over the shaping form from one direction. That is, while the shaping form 524 is shown as having convex contours on an outer surface thereof, such that the diameter of the form 524 varies along the axis A₁ of the tool 520-1, it should be understood that the form 524 may be implemented as a substantially straight cylinder in some examples.

In the examples of FIGS. 5A-5C, the induction coil 522 is embedded in the volume of the spacer form 524. Although the spacer form 524 is illustrated as having a hollow axial volume/channel 505, which may advantageously reduce the bulkiness, expense, and/or other complexity of the structural form 524, it should be understood that in some implementations the shape/form 524 may comprise a solid form having no channel or gap volume therein. The structural form 524 is shown as divided along an axial break 503.

The induction coil 522 may wind in a circumferential manner within the shaping form 524, producing a plurality of axially-offset winds/turns/coils in the coil 522. The diameter, thickness, spacing, and/or other aspect(s) of the configuration of the coil and winding may be designed to provide the desired magnetic field for producing induction heating in the workpiece 524 (and/or implant 510).

Magnetic field generation by the coil 522 may be facilitated by injecting electrical current into/through the coil. For example, the coil 522 may be electrically coupled and/or associated with an input lead 523-1, wherein electrical current may be injected into the coil via the lead 523-1 and pass through winds of the coil 522 before exiting the winds via an output lead 523-2, or vice versa. The coil 522 may advantageously wind axially in a distal direction and wind back proximally to the outlet lead/connector 523-2. Alternatively, the outlet lead 523-2 may be on an opposite end of the device 520, such that winding-back of the coil is not necessary. With the induction coil configured as shown, the coil 522 can act as a resistor (e.g., low-resistance resistor) in a closed electrical circuit. Although the winds of the coil 522 are shown as having axial spacing/gaps between adjacent winds of the coil 522, it should be understood that in some implementations, adjacent winds of the coil 522 may axially abut one another, such that little or no axial gap/spacing is present between adjacent winds.

As described in detail above, the shaping form/workpiece 524 may comprise ferromagnetic and/or non-ferromagnetic material, and may be configured to generate heat in response to a fluctuating magnetic field produced by the coil 522 as current flows through the coil. Therefore, as current flows through the coil 522, induction heating may be generated in the shaping form 524, wherein such heating is applied/transferred to the target implant 510 through conduction heating from contact with the shaping form 524, thereby heating the implant 510 to shape-set/anneal the memory-metal material thereof, as described in detail herein. Additionally or alternatively, the induction coil 522 may induce a current in the memory-metal frame 510 itself, thereby producing resistive heating in the struts of the frame 510 as a supplementation or alternative to the conductive heating from the shaping form 524.

The shaping form/workpiece 524 may comprise any material configured to support eddy currents induced by a changing magnetic field to generate heat. The efficiency of the induction heating of the workpiece 524 can be influenced at least in part by the particular magnetic properties of the material. Example materials that the workpiece 524 may comprise include stainless steel (e.g., ferritic and/or martensitic stainless steel), and/or other ferromagnetic materials, such as nickel, cobalt, iron, nickel-cobalt alloy, carbon steel, and the like. In some implementations, non-ferromagnetic materials may be used in the workpiece 524, such as aluminum, copper, brass, titanium, nitinol, gold, silver, and the like. Ferromagnetic materials may be preferable due to having a tendency to heat more quickly and efficiently in an induction heating system due to their relatively higher magnetic permeability, which serves to concentrate magnetic fields. However, non-ferromagnetic materials can also be effectively heated with induction in some examples, particularly for higher-frequency fields (e.g., radio-frequency induction). The selected material(s) for the workpiece 524 can affect the heating characteristics of the device based on their respective resistivity and magnetic permeability.

FIG. 5C shows an alternative implementation of the shaping form 524, wherein the shaping form 524 comprises multiple pieces. Although the shaping form 524 is illustrated in FIG. 5C as comprising first 524a and second 524b axially-offset pieces, it should be understood that the form 524 may have any modular/sectional configuration, such as any circumferential, radial, and/or axial modularity/sectioning.

In some implementations, where a multipiece/modular shaping form 524 is implemented, it may be necessary or desirable to utilize multiple induction coils integrated with the shaping form to facilitate the presence of coil windings in multiple separate pieces/segments, while allowing for disconnection/separation of the pieces. In such multi-coil implementations, such as is shown in FIG. 5C, multiple respective electrical current input and output leads 523 may be desired or necessary for the separate induction coils. For example, in the implementation of FIG. 5C, the first coil 522a, which is integrated with the first shaping piece 524a, includes one or more input and output leads 523a dedicated to the coil 522a, whereas the second coil 522b includes separate input/output leads 523b electrically coupled/integrated therewith and serving to provide electrical current that induces heating in the second shaping piece 524b.

As with any example disclosed herein induction coil 522 may serve to heat the workpiece 524 through induction heating, or additionally or alternatively, the coil 522 may heat the surrounding shaping workpiece 524 through resistive heating in the coil 522 and conductive transfer to the volume of the form 524 that surrounds the coil 522. That is, current in the coil 522, due to the resistance of the wire, may cause a resistive heating effect, wherein the proximity of the heated wire to the surrounding material of the shaping form 524 can heat the form 524, which in-turn can heat the memory-metal device 510 through conduction. Any induction or non-induction coil or conductor disclosed herein that is integrated with a shape-setting tool can be configured to produce heat through resistive heating that is transferable through conductive heating to a target memory-metal device. Use of coils and other conductors for combined induction heating and resistive heating can advantageously improve the rate of heat transfer to the target device and/or increase temperatures associated with shaping workpieces of examples of the present disclosure.

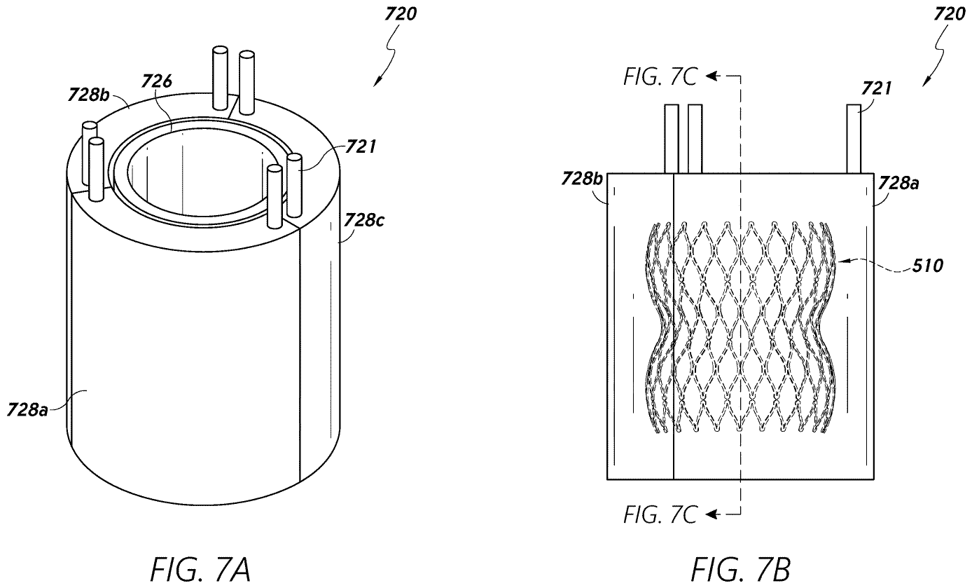

While some examples disclosed herein include induction coils that are integrated and/or embedded within one or more shaping form components/workpieces, it should be understood that induction coils associated with shape-setting tools of the present disclosure may be incorporated in the tool without embedding in the volume of the shaping form in some implementations. FIG. 6A shows a side view of a shaping workpiece 620 having an induction coil associated therewith in accordance with one or more examples. FIGS. 6B and 6C show side cross-sectional views of implementations of the shaping workpiece 620 of FIG. 6A, which has an induction coil 622 associated therewith, in accordance with one or more examples. As with certain other examples disclosed herein, the conductor/coil 622 is disposed within an outer diameter 609 of the shaping form(s) 624.

FIG. 6A shows the shape-setting tool 620 with a memory-metal implant 610, such as a stent/frame, or similar, disposed about a shaping form 624 of the tool 620. The shape-setting tool/workpiece 620 can include certain structural supporting and/or handling features, such as one or more extensions 621, which may be separate from and/or integrated with certain electrical conductor components utilized for supplying/passing current through an induction coil associated with the tool 620.

FIG. 6B shows a first example implementation 620-1 of the shape-setting tool/workpiece 620 shown in FIG. 6A. In the example of FIG. 6B, the shaping form/workpiece 624 has a single-piece implementation. In the examples of FIGS. 6A-6C, the induction coil 622 is disposed within an inner diameter 607 of the shaping form/workpiece 624. For example, the spacer form 624 can have a hollow axial volume/channel 605, wherein at least some of the winds of the coil 622 are disposed within the channel 605. The induction coil 622 may wind in a circumferential manner within the channel 605, producing a plurality of axially-offset winds/turns/coils in the coil 622. The diameter, thickness, spacing, and/or other aspect(s) of the configuration of the coil and winding may be designed to provide the desired magnetic field for producing induction heating in the workpiece 624 (and/or implant 610).

Magnetic field generation by the coil 622 may be facilitated by injecting electrical current into/through the coil. For example, the coil 622 may be electrically coupled and/or associated with an input lead 623-1, wherein electrical current may be injected into the coil via the lead 623-1 and pass through winds of the coil 622 before exiting the winds via an output lead 623-2. The coil 622 may advantageously wind axially in a distal direction and wind back proximally to the outlet lead/connector 623 - two. Alternatively, the outlet lead 623-2 may be on an opposite end of the device 620, such that winding-back of the coil is not necessary. Although the winds of the coil 622 are shown as having axial spacing/gaps between adjacent winds of the coil 622, it should be understood that in some implementations, adjacent winds of the coil 622 may axially abut one another, such that little or no axial gap/spacing is present between adjacent winds.

As described in detail above, the shaping form/workpiece 624 may comprise ferromagnetic and/or non-ferromagnetic material, and may be configured to generate heat in response to a fluctuating magnetic field produced by the coil 622 as current flows through the coil. Therefore, as current flows through the coil 622, induction heating may be generated in the shaping form 624, wherein such heating is applied/transferred to the target implant 610 through conduction heating from contact with the shaping form 624, thereby heating the implant 610 to shape-set/anneal the memory-metal material thereof, as described in detail herein. Additionally or alternatively, the induction coil 622 may induce a current in the memory-metal frame 610 itself, thereby producing resistive heating in the struts of the frame 610 as a supplementation or alternative to the conductive heating from the shaping form 624.

FIG. 6C shows an alternative implementation of the shaping form 624, wherein the shaping form 624 comprises multiple pieces. Although the shaping form 624 is illustrated in FIG. 6C as comprising first 624a and second 624b axially-offset pieces, it should be understood that the form 624 may have any modular/sectional configuration, such as any circumferential, radial, and/or axial modularity/sectioning.

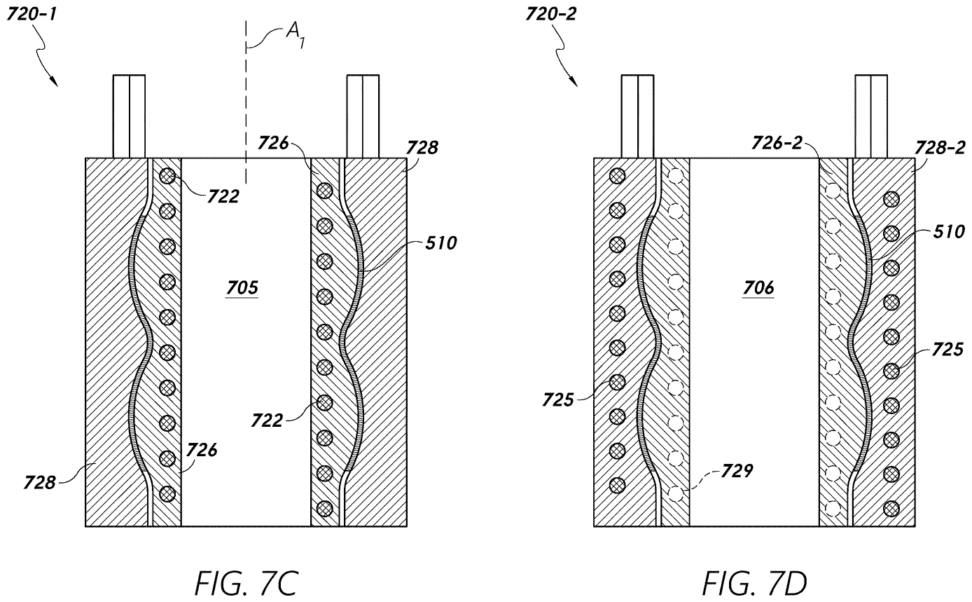

In some multi piece shaping form implementations, the shaping form includes outer/exterior pieces configured to constrain and/or shape the target device from a radially-outside position. FIGS. 7A and 7B show perspective and side views, respectively, of a multi-piece shaping workpiece 720 in accordance with one or more examples. The shape-setting tool 720 includes one or more inner shaping and/or support form pieces 726, as well as one or more outer shaping/support form pieces 728. In some implementations, the outer shaping component 728 comprises a plurality of pieces, as shown in FIG. 7A, wherein each piece may have associated therewith separate structural and/or electrical components/leads 721.

The outer shaping pieces 728 can be circumferentially arranged and can have a curved shape configured to form an at least partially cylindrical outer container around the target implant 510 (see FIG. 7B) to hold and/or shape the implant 510 from the radially-outside dimension. The image of the side view of FIG. 7B shows example positioning of the target implant/device 510 disposed within the containment of the outer forms 728.

FIG. 7C shows a side cross-sectional view of a first implementation 720-1 of the multi-piece shaping workpiece 720 shown in FIGS. 7A and 7B having an induction coil 722 integrated with one or more interior pieces 726 thereof in accordance with one or more examples. In the example of FIG. 7C, the induction coil 722 is integrated and/or at least partially embedded within the inner shaping component 726 of the tool 720-1. With respect to examples disclosed herein, an ‘inner’ shaping component/form may refer to a shaping form configured to be positioned radially within a memory-metal implant with respect to an axis of the implant. For example, with respect to a cylindrical stent, an inner shaping component/form may be disposable at least partially within an inner diameter of the cylinder/tube, such as within the flow channel of a target stent implant. The shape-setting tool 720 further includes an outer shaping form/component 728, which may comprise one or more pieces or segments (e.g., circumferentially divided/separated pieces).

The inner 726 and outer 728 shaping forms/components may be configured to sandwich at least a portion of the implant device 510 therebetween, which may advantageously produce a shaping effect on the target implant 510 in multiple directions. In some implementations, the outer shaping component 728 comprises a unitary cylindrical component configured to slide over the implant device 510, wherein the implant device 510 is disposed or disposable on a cylindrical inner shaping cylinder. In some implementations, one or both of the inner 726 and/or outer 728 shaping forms may be axially separable/divided into multiple segments that are separable from one another in the axial dimension. With the induction coil 722 being associated with the inner shaping form 726, the coil 722 may be configured to direct magnetic fields in a manner that primarily heats through induction heating in/by the inner component 726, which in turn heats the target implant 510 through conduction. Additionally or alternatively, the induction coil 722 may generate heat in the outer component 728, such that the outer component 728 can serve to heat the implant device 510 through conduction from the outer diameter thereof. In some implementations, both the inner 726 and outer 728 shaping forms are heated in response to operation of the induction coil 722, thereby producing a heating effect on the target implant 510 from both inner-diameter contact and outer-diameter contact, which can advantageously increase the speed of heating of the implant device 510 to the desired shape-setting/annealing temperature.

Whereas the example illustrated in FIG. 7C shows the inductor coil 722 integrated with only the inner component 726 of the shaping tool 720-1, FIG. 7D shows an example in which an induction coil 725 is integrated and/or embedded with/within at least a portion of the outer shaping form/component 728-2. However, it may be desirable for the coil 725/722 to be integrated with the inner shaping form 726 in examples in which the outer shaping form 728 comprises multiple circumferentially-divided/arranged pieces.

With the induction coil 725 being associated with the outer shaping form 728-2, the coil 725 may direct magnetic field in a manner that primarily heats through induction heating of the outer component 728-2, which in turn heats the target implant 510 through conduction. Additionally or alternatively, the induction coil 725 may generate heat in the inner component 726-2, such that the outer component can serve to heat the implant device 510 through conduction from the inner diameter thereof. In some implementations, both the inner 726-2 and outer 728-2 shaping forms are heated in response to operation of the induction coil 725, thereby producing a heating effect on the target implant 510 from both inner-diameter contact and outer-diameter contact.

In some implementations, both the outer 728-2 and inner 726-2 shaping components have induction coil features integrated/associated therewith. In such implementations, conductive heating of the shaping workpieces 726-2, 728-2 can be relatively quick compared to coil integration in just one of the inner or outer components. FIG. 7D shows optional inner conduction coil features 729 that may be implemented in combination with the outer conduction coil features 725. In such a configuration, either or both of the inner 729 and outer 725 coil features may produce/generate heating in either or both of the inner 726-2 and/or outer 728-2 shaping forms/workpieces. In some implementations, only one of the inner 726-2 or outer 728-2 shaping components is ferromagnetic or conductive in a manner as to be responsive to the alternating magnetic fields to produce thermal energy. That is, only one of the inner 726-2 or outer 728-2 shaping components may become heated in response to operation of the inductive coil(s) and perform heating of the target device 510.

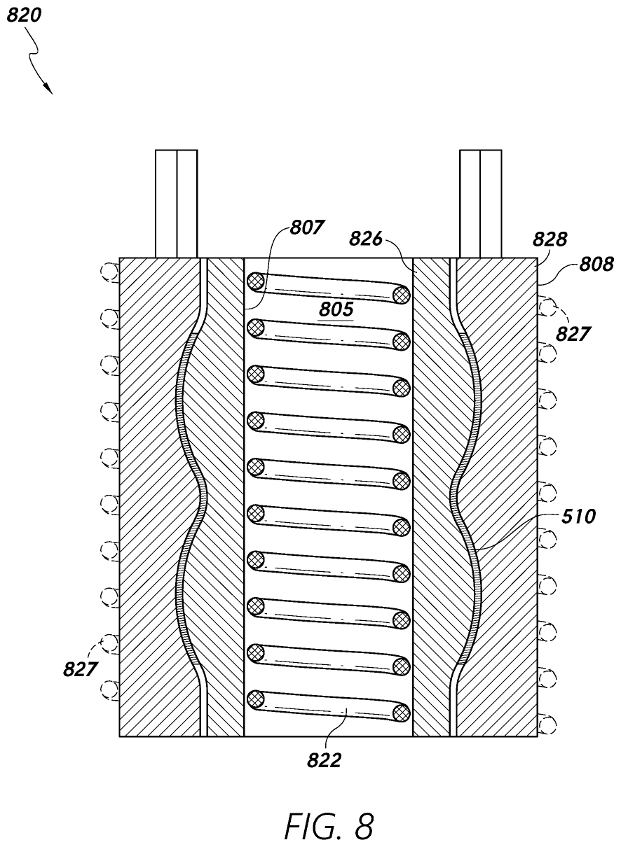

FIG. 8 shows a side cross-sectional view of a multi-piece shaping workpiece 820 having an induction coil 822 associated with an inner 807 and/or outer 808 diameter thereof in accordance with one or more examples. In the example of FIG. 8, the induction coil 822 is disposed within an inner diameter 807 of an inner shaping form/workpiece 826 of the tool 820. For example, the spacer form 826 can have a hollow axial volume/channel 805, wherein at least some of the winds of the coil 822 are disposed within the channel 805. The induction coil 822 may wind in a circumferential manner within the channel 805, producing a plurality of axially-offset winds/turns/coils in the coil 822. The diameter, thickness, spacing, and/or other aspect(s) of the configuration of the coil and winding may be designed to provide the desired magnetic field for producing induction heating in the inner 826 and/or outer 828 workpiece(s) (and/or implant 510).

In some implementations, as an alternative, or in addition, to the inner coil 822, the shape-setting tool 820 may comprise one or more coils disposed and/or wrapped around the outer diameter/exterior 808 of the shape-setting tool 820. For example, FIG. 8 shows an optional coil 827 disposed about the outer diameter 808 of the outer shaping form 828. Such outer coil 827 may generate alternating magnetic fields that produces heating in either or both of the outer 828 or inner 826 shaping workpieces to heat the target implant 510.

With respect to shape-setting tools as described herein that comprise multipiece inner and/or outer shaping forms/components, where such pieces provide circumferential breaks/divisions with respect to an axis of the tool, induction coils wrapped circumferentially within the shaping pieces may be unsuitable due to the undesirability of cutting or breaking the coils at the breakpoints/divisions of the adjacent shaping components/pieces with which they are integrated or associated. Therefore, it can be desirable to implement induction coils in such tools in a manner such that the coils do not wrap circumferentially around the axis of the tool, but rather lie in an alternative plane/dimension.

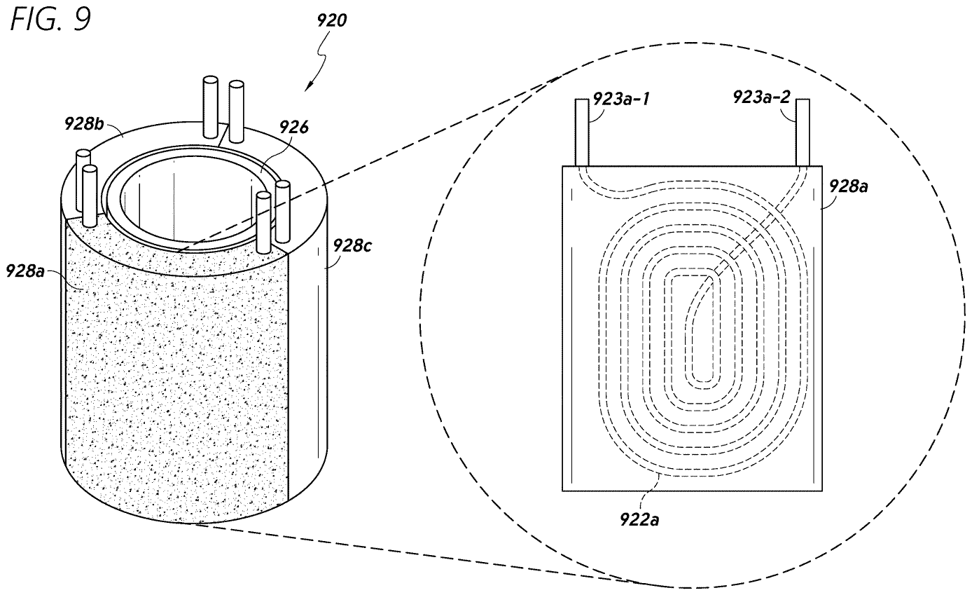

FIG. 9 shows perspective and side views of a piece 928a of a multipiece shaping workpiece 920 that has a dedicated/corresponding induction coil portion 922a associated therewith in accordance with one or more examples. The example of FIG. 9 includes three circumferentially-arranged shaping pieces 928a, 928b, 928c. In the example of FIG. 9, the shape-setting tool 920 includes one or more coils, including the coil 922a, which are integrated and/or associated with a single respective segmented/modular piece of the inner 926 or outer 928 shaping form(s). For example, as shown in the detailed image on the right of FIG. 9, the coil 922a, which may have associated therewith input and output connectors 923, as well as a plurality of coil winds, may be integrated/embedded in a single circumferential piece/segment 928a of the shaping form 928. For example, the coil 922a includes a plurality of complete winds in the piece 928a in the illustrated implementation of FIG. 9. In some implementations, as in the implementation illustrated in FIG. 9, the coil 922a may include a plurality of winds that lie at least partially in a common plane. For example, the plane in which the coil 922a is wound may be a curved circumferential plane having a radius of curvature corresponding to a radial segment of the shaping piece 928a. That is, at least some of the winds of the coil 922a may lie in a common curved plane that runs through the shaping piece/segment 928a with which is associated.

Although only the coiled line/wire 922a is visibility illustrated in FIG. 9, it should be understood that the shape-setting tool 920 may include respective coils for each of a plurality of shaping pieces/segments. For example, with respect to the illustrated implementation of FIG. 9, the tool 920 may include three separate coils associated respectively with the three circumferential outer shaping pieces/segments 928. Although illustrated in the outer shaping piece 928a, it should be understood that coils, such as the coil 922a, may be implemented additionally or alternatively in one or more pieces/segments of the inner shaping form 926.

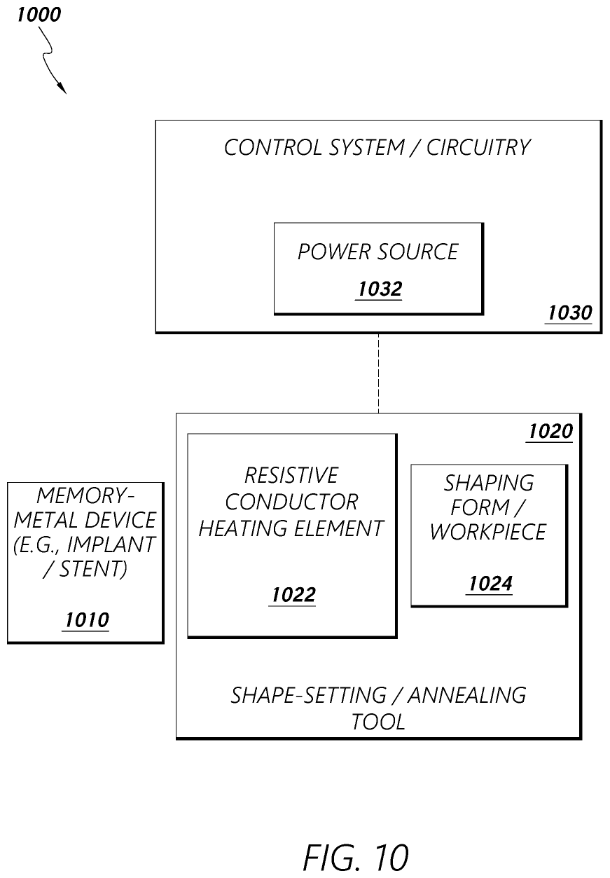

FIG. 10 is a block diagram of a resistive heating system 1000 for memory-metal shape-setting in accordance with one or more examples. The system 1000 is designed to implement resistive heating using a conductor heating element 1022, which may be shaped to produce a shaping effect in addition to heating in some examples. For example, the resistive conductor heating element 1022 and shaping form 1024 may be formed of a unitary structure/form (e.g., the conductor) in some examples.

As an alternative to salt bath immersion heating and similar processes for memory-metal shape-setting/annealing, the system 1000 is designed to use a shaping tool/workpiece 1024 having an integrated/associated conductor (e.g., coil) 1022 configured to heat in response to electrical current and radiate/conduct such heat for annealing/shape-setting a target memory-metal device 1010 (e.g., stent) relatively quickly, safely, cleanly, and with less space requirement and equipment complexity compared to salt bath annealing.

Generally, the process of resistive heating requires a conductor that is used to conduct electrical current, wherein the resistance of the conductor generates heat as voltage is dissipated in the conductor. Certain examples of the present disclosure advantageously combine the shaping tool/mandrel 1024, which serves as a shaping workpiece, and the conductor 1022 into a single unit/device, thereby providing heating for shape-memory shape-setting/annealing and shaping in one device/structure 420. The conductor heat source 1022 is advantageously configured to generate sufficient heat in the workpiece 1024 to metallurgically transform the memory metal part 1010 to induce a new shape. The implant 1010 may be any type of shape-memory implant, such as a component of a prosthetic valve (e.g., mitral), blood vessel or heart valve docking device (e.g., pulmonary valve dock), valve repair device/clip, delivery system, or any other device.

Some of the disclosed resistive-heating solutions can combine the shape/function of a shape-setting tool/mandrel with a resistive conductor to produce a construction that promotes relatively rapid processing of memory-metal shape-set components. The shape-setting workpiece 1024 can have an at least partially cylindrical shape, for example. The resistive/conductive heating tool 1020 serves a dual purpose, to both heat and shape the memory-metal part 1010 simultaneously.

The heating system 1000 includes the resistive heating element 1022, which is powered by a power supply 1032, which may comprise a direct-current or alternating-current voltage/current source. The power source 1032 can include an electrical outlet, battery, or generator. Generally, the amount of heat generated in the conductor 1022 may be proportional to the square of the current flowing through the conductor 1022 and the resistance of the material of the conductor 1022.

The conductive heating element 1022 can comprise any suitable conductor, such as tungsten, stainless steel, copper, aluminum, ceramic, nichrome, kanthal or other iron alloy, or the like. The conductor 1022 may be shaped and positioned to direct conducted and/or radiated heat within and/or toward the shaping form/workpiece 1024 to effectively conduct thermal energy to the target device 1010. The shape and/or position of the conductor 1022 can at least partially determine the distribution of thermal energy and influence the heating pattern caused thereby. For example, the resistance, thickness, width, and/or shape of the conductor 1022 can be designed to produce the desired targeting of the heating.

The conductor heating element 1022 comprises a resistive component that converts electrical energy into heat. Therefore, it may be desirable to utilize, for the element 1022, a material with relatively high electrical resistance, such as nichrome wire or ceramic. The heating element 1022 is advantageously designed to withstand temperatures generated during the heating process that are sufficiently high to shape-set the target device 1010. The resistive heating element 1022 is part of a closed electrical circuit that runs through the heating element 1022, wherein the closed circuit allows the flow of electrical current through the heating element 1022, which in-turn converts the electrical current into heat. The heating element 1022, which can advantageously have relatively high electrical resistance, acts as a resistor in the circuit. Therefore, when electrical current passes through the heating element 1022 (e.g., wire), it encounters resistance, and this resistance causes the electrical energy to be converted into heat. The heat generated by the heating element 1022 is then transferred to the shaping form 1024 (if separate from the heating element 1022) and/or to the target device 1010.

The power source 1032 may be associated with control system circuitry 1030 configured to regulate and/or monitor the heating process, and may provide certain temperature-control and safety features. The control system/circuitry 1030 can comprise one or more temperature sensors, thermostats, power controllers, and/or control mechanisms to maintain the desired temperature and/or power level.

The shaping form/workpiece 1024 can comprise an electrically- and/or thermally-conductive material, and may (or may not) be formed of the resistive conductor itself, at least in part. In some implementations, the workpiece 1024 comprises at least one of steel, aluminum, copper, brass, or other conductive metal/alloy.

The shape-setting tool 1020 can comprise certain thermally-reflective and/or insulating features configured to prevent heat loss and/or protect against electrical hazards. Such features can comprise ceramic, mica, refractory materials, or the like, which may be designed to enclose the heating element on one or more sides and/or direct generated heat in the direction of the target 1010.

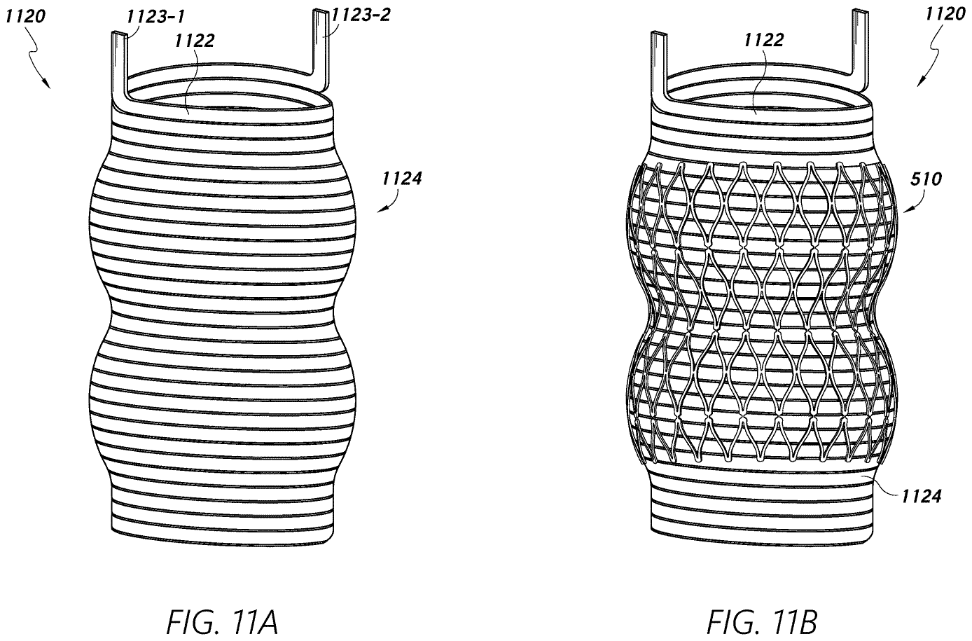

FIGS. 11A and 11B show a resistive heating workpiece 1120 formed of one or more electrical conductors in accordance with one or more examples. As with other examples disclosed herein, the resistive heating workpiece 1120 may be configured to provide both shaping and heating functionality for shape-setting a target device 510, such as a nitinol stent or other memory-metal implant device. For example, the resistive conductor heating element 1122 may be injected with current at an input/source terminal 1123-1, wherein an output terminal 1123-2 provides a sink/outlet for the electrical current, thereby enabling current through the conductor 1122 in a closed circuit. The conductor 1122 may be shaped with respect to an outer diameter thereof, or other portion, to provide a desirable shape for the target memory-metal device 510, which may be placed on the shape-setting tool 1120, as shown in FIG. 11B.

In addition to providing the shaping effect desired for the target device 510, the conductor 1122 may generate heat in response to electrical current flowing therethrough as a function of the resistance of the conductor 1122. Such resistive heating can be transferred to the target device 510 through conduction due to the physical proximity of the device 510 to the heated conductor 1122, thereby shape-setting the memory-metal device 510 to the shape forced/held by the shaping form of the conductor 1122. Alternatively, in some implementations, an intermediate shaping structure/form is used to shape and heat the target device 510, wherein the coil heats the intermediate structure/form and the intermediate structure (e.g., thermally-conductive volume having the desired shaping shape) in-turn heats the target device 510 through conductive and/or radiative thermal transfer. In such examples, the conductor 1122 may be embedded/integrated in/with the intermediate structure/form.

In some implementations, the conductor 1122 may be wound in winds of a coil, as illustrated, which may be similar in certain respects to various induction coils disclosed herein with respect to the configuration/shape thereof. Alternatively and/or additionally, the conductor 1122 may be arranged in any other configuration that forms the desired shaping form and/or allows for electrical current to flow into and out of the length of the conductor 1122, thereby facilitating resistive heating in the conductor 1122.

The configuration of the conductor 1122 may facilitate manually or otherwise manipulating the shape thereof to become radially compressed to facilitate placement over the conductor form 1124 of the memory-metal implant/device 510. For example, the conductor 1122 itself may be shape-set to the desired shape of the form 1124, such that deformation from such shape stores energy in the device 1120 that returns the form 1124 to the expanded shape after compression forces are removed/decreased. In some implementations, the conductor 1122 is configured as winds of a coil having a spring-like configuration, which may enable radial compression or reduction in dimension to facilitate placement thereon of the target device 510. For example, winding or stretching of the conductor form 1124 may produce a reduction in diameter in one or more portions thereof, thereby reducing the profile of the tool 1124 and allowing for placement thereon of the target device 510.

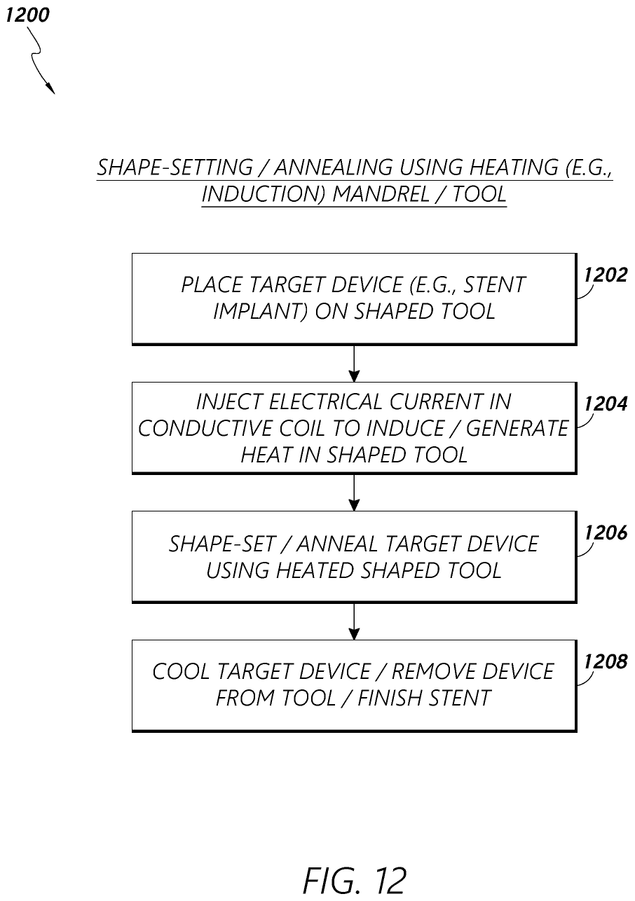

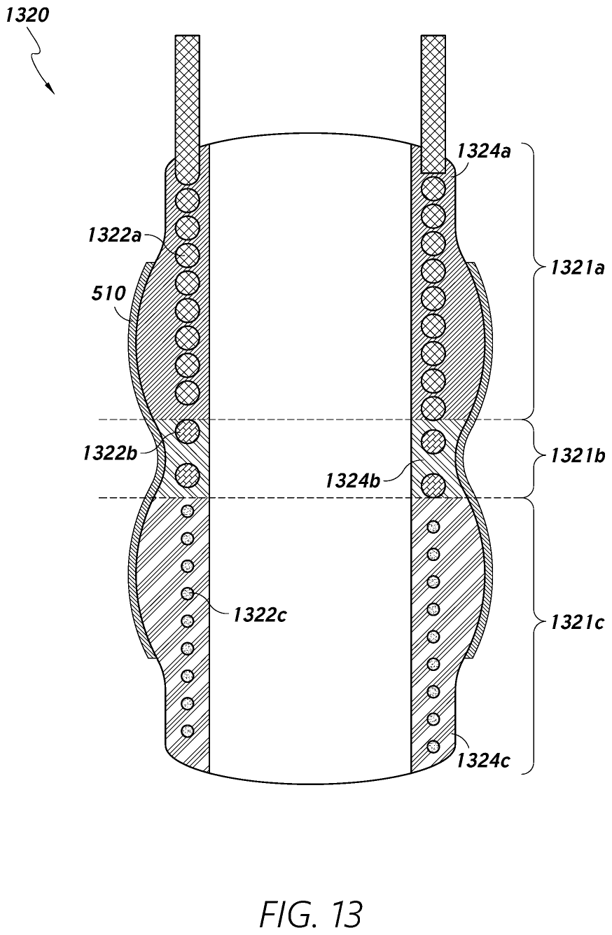

FIG. 12 is a flow diagram illustrating a process 1200 for heating a memory metal device using a heating tool in accordance with one or more examples. Compared to certain other solutions relying solely on conductive heating through immersion in a heated bath, which can be relatively expensive and slow, solutions of the present disclosure, as described in a subset of implementations relating to the process 1200 of FIG. 12, can produce quicker and cheaper shape-setting/annealing of memory-metal device (e.g., nitinol stents). Furthermore, while immersion-bath shape-setting generally produces parts with uniform metallurgical properties, examples of the present disclosure can be designed to leverage induction and/or resistive heating in a selective manner as to produce parts with variable metallurgical properties across the geometry (see, e.g., FIG. 13 and associated description), which can provide unique/customized part functionality/characteristics.

At block 1202, the process 1200 involves placing a target memory-metal device (e.g., such stent implant or the like) on a shape-setting tool having a shaped form and one or more integrated conductor elements configured to produce a heating effect in the shaping form. The step(s) of block 1202 may involve physically-coupling the target device to the shape-setting tool in any manner as to cause the device to conform at least in part to a shape of at least a portion of the shape-setting tool. In some examples, the conductive elements comprise one or more conductive coils integrated with the shaping form and/or forming the shaping form. In some implementations, the conductor(s) are disposed within and/or without the shaped tool, but not embedded in the form/volume thereof.

At block 1204, the process 1200 involves injecting electrical current into the conductive coil(s)/conductor(s) to induce or otherwise generate heat in the shaped form/tool (e.g., inductive heating workpiece). For example, heat may be generated in the shaped tool through inductive transfer from the conductive coil/conductor. Additionally or alternatively, heat may be generated in the conductor/coil itself due to resistive heating effects from the resistance thereof, wherein such heat may be transferred through conduction to the shaping tool and/or target memory-metal device.

At block 206, the process 1200 involves shape-setting/annealing the target memory-metal device using the heated shaped tool. The heat in the shaped tool may be transferred to the target memory-metal device through conduction, wherein sufficient heat is generated in the shaped tool to produce a shape-setting effect in the memory-metal material of the target device, as described in detail herein.

The electrical conductor(s) and/or shaping tool/workpiece may be designed to variably heat the shaping form to achieve multiple desired metallurgical properties in predictable areas of the target device. For example, the conductor (e.g., coil) may be modified to heat separate sections of a part to different temperatures and/or for different heating periods, which may be achieved by, for example, changing the susceptibility of the shaped workpiece, and/or by changing the design of the conductor(s) in different areas thereof to deliver different amounts of induced/generated thermal energy to the different parts of the target device. Various advantages may be associated with selective/variable heating using a single tool as described herein, including the ability to achieve different/varying mechanical properties in a target device dependent on how it has been heated. Selective/variable heat treatment may also be advantageous for shape-setting parts made from materials other than nitinol.

At block 1208, the process 1200 involves cooling the target memory-metal device (e.g., stent/implant), removing the device from the shape-setting tool, and/or finishing the device in accordance with one or more processes to produce a suitable final product. Cooling of the target device may take place while the implant remains disposed on the shaping tool and/or after removal therefrom.

The shape-setting/annealing process 1200 can be preferable to molten-salt-bath shape-setting/annealing as providing improved space, energy, and/or cost in comparison. Furthermore, some implementations of the process 1200 can provide for multiple metallurgical properties within a single part, which is not achievable in molten-salt-bath processes.

FIG. 13 shows a heating workpiece 1320 configured to produce memory-metal shape-setting variability in accordance with one or more examples. Various areas or portions of the shape-setting device/tool 1320 may have different/varying material and/or conductor (e.g., coil) features/characteristics associated therewith, which may produce variation in the magnetic fields produced by the coils and/or in the heating effect of the shaped form 1324. For example, for illustration purposes, FIG. 13 shows three axial portions/segments 1321a, 1321b, 1321c of the device 1320, each having different conductor and/or material characteristics to produce potentially different heating effects in different areas of the device 1320. While three separate axial segments 1321a and 1321b, 1321c are shown, it should be understood that any example of the present disclosure may have any number or configuration of portions of shaping form material and/or electrical conductor (e.g., coil) design or configuration to produce any desirable variable heating in the shape-setting device, including circumferentially- and/or axially-offset variable portions.

Each of the separate portions/segments 1321 of the tool 1320 may have different or similar shaping form material composition compared to one or more other portions/segments. For example, the first segment 1321a may have associated therewith a shaping form segment 1324a that comprises a first material, whereas the second 1321b and third 1321c segments may have respective shaping form segments 1324b, 1324c that have different or similar material composition compared to the segment 1324a. Material variability may include higher concentrations of certain constituents of the various material portions to produce different ferromagnetic, electrical conductivity, and/or thermal conductivity properties to produce the desired heating effect for the target device 510 in the corresponding area/portion thereof.

In addition, the conductor/coil 1322 may have varying features between one or more of the different segments 1321. For example, in the first segment 1321a, the coil 1322a may have different wire thickness and/or spacing features between adjacent winds of the coil 1322a than one or both of the other segments 1321b, 1321c. For example, the segment 1321b shows wire coil having greater axial spacing between adjacent winds of the coil compared to the segment 1321a, whereas the coiled portion 1322c includes thinner wire than the wire in the first 1321a and second 1321b segments.

The variable conductor and/or workpiece material characteristics of the tool 1320 can provide various benefits compared to, for example, immersion conduction heating/annealing (e.g., salt bath), which produces parts with uniform metallurgical properties. The ability to control the specific application of heat to targeted areas/portions of a target device (e.g., nitinol implant) can advantageously produce variable shape-setting properties in a single device. Examples of such application may include heating different areas across the geometry of a memory metal part for different periods of time and/or to different temperatures. The tool 1320 can advantageously provide variable heating corresponding to different areas of the tool, and thus produce location-dependent metallurgical properties in the target component, while achieving the desired shape, in a single process. The variability of heat application to different areas of a memory-metal component can produce portions of the component having different strength in the austenite form, different activation temperature, or other properties. Furthermore, variable heating using a device like the tool 1320 can be used to apply different intensity and/or duration of heat to portions of the target component that have different wire thicknesses as a means to produce uniform properties across target geometries that have variable wire/material thickness.

For example implementations of a variable-heating device as in FIG. 13 that utilizes induction coil heating, such induction heating can provide a relatively precise method of heating, allowing for the application of very specific temperatures to the target. Furthermore, by tuning the inductor coil so certain parts are more active than others, different heating in different regions is enabled.

Changing the temperature of heating of selected areas of a target device, thereby varying the respective transition temperature, can affect the yield strength upper plateau, such that the amount of force that the material exerts when deformed varies across the geometry. Therefore, the target 510 can be shape-set such that it is relatively really rigid, or soft/subtle, in different areas.

### ADDITIONAL EXAMPLES

Provided below is a list of examples, each of which may include aspects of any of the other examples disclosed herein. Furthermore, aspects of any example described above may be implemented in any of the numbered examples provided below.

Example 1: A shape-setting device comprising an electrical conductor, and a shaping form configured to have a target memory-metal device physically coupled thereto to hold at least a portion of the target memory-metal device in a shape conforming to at least a portion of the shaping form.

Example 2: The shape-setting device of any example herein, in particular example 1, wherein the electrical conductor comprises an inductor coil.

Example 3: The shape-setting device of any example herein, in particular example 2, wherein the inductor coil is embedded at least partially within the shaping form.

Example 4: The shape-setting device of any example herein, in particular example 2, wherein the inductor coil is disposed within a channel of the shaping form.

Example 5: The shape-setting device of any example herein, in particular example 2, wherein the inductor coil wraps circumferentially about an axis of the shaping form.

Example 6: The shape-setting device of any example herein, in particular example 2, wherein the shaping form comprises ferromagnetic material configured to generate heat in response to a magnetic field produced by the inductor coil.

Example 7: The shape-setting device of any example herein, in particular example 1, wherein the shaping form comprises an inner portion and an outer portion configured to hold the target memory-metal device between the inner and outer portions of the shaping form.

Example 8: The shape-setting device of any example herein, in particular example 7, wherein the electrical conductor is embedded at least partially within the inner portion of the shaping form.

Example 9: The shape-setting device of any example herein, in particular example 7, wherein the electrical conductor is embedded at least partially within the outer portion of the shaping form.

Example 10: The shape-setting device of any example herein, in particular example 7, wherein the outer portion of the shaping form comprises multiple circumferential pieces.

Example 11: The shape-setting device of any example herein, in particular example 10, wherein the electrical conductor comprises a plurality of complete winds in one of the multiple circumferential pieces of the outer shaping portion.

Example 12: The shape-setting device of any example herein, in particular example 1, wherein the electrical conductor is configured to generate resistive heating in response to electrical current flowing therethrough.

Example 13: The shape-setting device of any example herein, in particular example 12, wherein the shaping form is formed of the electrical conductor.

Example 14: The shape-setting device of any example herein, in particular example 12, wherein the shaping form comprises a thermally-conductive volume.

Example 15: The shape-setting device of any example herein, in particular example 14, wherein the electrical conductor is embedded at least partially within the thermally-conductive volume.

Example 16: The shape-setting device of any example herein, in particular example 14, wherein the electrical conductor is disposed within an inner channel of the shaping form.

Example 17: The shape-setting device of any example herein, in particular example 14, wherein the electrical conductor and shaping form are configured to sandwich the target memory-metal device.

Example 18: The shape-setting device of any example herein, in particular example 17, wherein the electrical conductor is disposed radially outside of the shaping form.

Example 19: The shape-setting device of any example herein, in particular example 1, wherein the shape-setting device is configured to, in response to electrical current in the electrical conductor, produce variable heating in different areas of the shape-setting device.

Example 20: The shape-setting device of any example herein, in particular example 19, wherein at least one of the electrical conductor or the shaping form has variable physical characteristics to produce the variable heating.

Example 21: The shape-setting device of any example herein, in particular example 20, wherein the electrical conductor comprises an inductive coil, and the inductive coil has at least one of variable conductor thickness or variable wind spacing along a length of the coil.

Example 22: The shape-setting device of any example herein, in particular example 20, wherein the shaping form has at least one of variable material composition or variable thickness over a portion of a volume of the shaping form.

Example 23: A shape-setting device comprising a cylindrical ferromagnetic workpiece, and an inductive coil forming a plurality of winds about an axis of the workpiece, the inductive coil being disposed at least partially within an outer diameter of the workpiece.

Example 24: The shape-setting device of any example herein, in particular example 23, wherein the workpiece comprises, one or more inner shaping forms, and one or more outer shaping forms, wherein the inductive coil is disposed within at least one of the one or more inner shaping forms, the one or more outer shaping forms, or an axial channel defined by the one or more inner shaping forms.

Example 25: The shape-setting device of any example herein, in particular example 24, wherein the one or more outer shaping forms comprises a plurality of circumferentially-arranged shaping pieces, and the inductive coil is disposed within one of the plurality of circumferentially-arranged shaping pieces.

Example 26: The shape-setting device of any example herein, in particular example 25, wherein the plurality of winds lie in a plane that is curved about the axis of the workpiece.

Example 27: The shape-setting device of any example herein, in particular example 23, wherein the inductive coil is configured to generate resistive heat in response to electrical current therein, and conduct the heat to the workpiece.

Example 28: A method of shape-setting a memory-metal device, the method comprising physically-coupling a memory-metal device to a shaped workpiece, injecting electrical current through a conductor associated with the shaped workpiece to heat the shaped workpiece, and conducting thermal energy from the shaped workpiece to the memory-metal device to shape-set at least a portion of the memory-metal device to a shape conforming to at least a portion of the shaped workpiece.

Example 29: The method of any example herein, in particular example 28, wherein the conductor comprises an inductor coil, and the electrical current is an alternating current.

Example 30: The method of any example herein, in particular example 29, wherein said heating the shaped workpiece comprises generating an alternating magnetic field around the conductor using the alternating current, inducing an eddy current in the shaped workpiece using the alternating magnetic field, and generating heat in the shaped workpiece using the eddy current and electrical resistivity of the shaped workpiece.

Example 31: The method of any example herein, in particular example 30, wherein the shaped workpiece comprises ferromagnetic material.

Example 32: The method of any example herein, in particular example 30, wherein the shaped workpiece comprises non-ferromagnetic material.

Example 33: The method of any example herein, in particular example 28, wherein said heating the shaped workpiece comprises heating the conductor using the electrical current, and conducting heat to the shaped workpiece from the conductor through physical contact.

Example 34: The method of any example herein, in particular example 28, wherein the conductor is embedded at least partially within a volume of the shaped workpiece.

Example 35: The method of any example herein, in particular example 28, wherein the conductor is disposed within an axial channel of the shaped workpiece.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A shape-setting device comprising:
an electrical conductor; and
a shaping form configured to have a target memory-metal device physically coupled thereto to hold at least a portion of the target memory-metal device in a shape conforming to at least a portion of the shaping form.

2. The shape-setting device of claim 1, wherein the electrical conductor comprises an inductor coil.

3. The shape-setting device of claim 2, wherein the inductor coil is embedded at least partially within the shaping form.

4. The shape-setting device of claim 2, wherein the inductor coil is disposed within a channel of the shaping form.

5. The shape-setting device of claim 2, wherein the inductor coil wraps circumferentially about an axis of the shaping form.

6. The shape-setting device of claim 2, wherein the shaping form comprises ferromagnetic material configured to generate heat in response to a magnetic field produced by the inductor coil.

7. The shape-setting device of claim 1, wherein:
the shaping form comprises an inner portion and an outer portion configured to hold the target memory-metal device between the inner and outer portions of the shaping form; and
the electrical conductor is embedded at least partially within the inner portion of the shaping form or the outer portion of the shaping form.

8. The shape-setting device of claim 7, wherein:
the outer portion of the shaping form comprises multiple circumferential pieces; and
the electrical conductor comprises a plurality of complete winds in one of the multiple circumferential pieces of the outer portion of the shaping form.

9. The shape-setting device of claim 1, wherein the electrical conductor is configured to generate resistive heating in response to electrical current flowing therethrough.

10. The shape-setting device of claim 9, wherein the shaping form is formed of the electrical conductor.

11. The shape-setting device of claim 9, wherein:
the shaping form comprises a thermally-conductive volume; and
the electrical conductor is embedded at least partially within the thermally-conductive volume.

12. The shape-setting device of claim 1, wherein the shape-setting device is configured to, in response to electrical current in the electrical conductor, produce variable heating in different areas of the shape-setting device.

13. The shape-setting device of claim 12, wherein at least one of the electrical conductor or the shaping form has variable physical characteristics to produce the variable heating.

14. The shape-setting device of claim 13, wherein:
the electrical conductor comprises an inductive coil; and
the inductive coil has at least one of variable conductor thickness or variable wind spacing along a length of the inductive coil.

15. The shape-setting device of claim 13, wherein the shaping form has at least one of variable material composition or variable thickness over a portion of a volume of the shaping form.

16. A method of shape-setting a memory-metal device, the method comprising:
physically-coupling a memory-metal device to a shaped workpiece;
injecting electrical current through a conductor associated with the shaped workpiece to heat the shaped workpiece; and
conducting thermal energy from the shaped workpiece to the memory-metal device to shape-set at least a portion of the memory-metal device to a shape conforming to at least a portion of the shaped workpiece.

17. The method of claim 16, wherein:
the conductor comprises an inductor coil; and
the electrical current is an alternating current.

18. The method of claim 17, wherein said heating the shaped workpiece comprises:
generating an alternating magnetic field around the conductor using the alternating current;
inducing an eddy current in the shaped workpiece using the alternating magnetic field; and
generating heat in the shaped workpiece using the eddy current and electrical resistivity of the shaped workpiece.

19. The method of claim 16, wherein said heating the shaped workpiece comprises:
heating the conductor using the electrical current; and
conducting heat to the shaped workpiece from the conductor through physical contact.

20. The method of claim 16, wherein the conductor is embedded at least partially within a volume of the shaped workpiece.
